(19) United States
(12) Patent Application Publication
(10) Pub. No.: US 2021/0015009 A1
(43) Pub. Date: Jan. 14, 2021

(54) EARTHING POLES FOR GENERATING A HEALTHY ENERGY VORTEX

(71) Applicants: John A. Robbins, Costa Mesa, CA (US); Todd Evan Fligner, Laguna Niguel, CA (US)

(72) Inventors: John A. Robbins, Costa Mesa, CA (US); Todd Evan Fligner, Laguna Niguel, CA (US)

(21) Appl. No.: 16/924,999

(22) Filed: Jul. 9, 2020

Related U.S. Application Data
(60) Provisional application No. 62/921,803, filed on Jul. 9, 2019.

(51) Int. Cl. H05K 9/00

(52) U.S. Cl. CPC H05K 9/0064; H05K 9/009

(57) ABSTRACT

The present invention is directed to a system for generating a healthy energy vortex within a space through an earthing effect. The system may comprise a plurality of copper-clad earthing poles encompassing a perimeter of an area. Each earthing pole of the plurality of earthing poles may be completely submerged underground, may contact a plurality of healthy negative ions underground, and may not contact a separate structure. Each adjacent pair of earthing poles may be separated by a distance suitable for generating the healthy energy vortex through the earthing effect. Each earthing pole may further comprise a cap of grounding enhancement material and a wire connecting the earthing pole to adjacent earthing poles. The system may further comprise a plurality of magnet pairs disposed underground outside of the perimeter of the area encompassed by the plurality of earthing poles.

### CROSS-REFERENCES TO RELATED APPLICATIONS

This application is a non-provisional and claims benefit of U.S. Provisional Application No. 62/921,803 filed Jul. 9, 2019, the specification of which is incorporated herein in its entirety by reference.

### FIELD OF THE INVENTION

The present invention is directed to a system for generating a healthy energy vortex within a space through an earthing effect.

### BACKGROUND OF THE INVENTION

Electromagnetic frequency “EMF” is increasing at an alarming rate in many parts of the earth, especially now with the addition of 5G products emerging throughout the world. This increase in saturation of microwave energy is adding to the damage of humans, animals, crops, and all vegetation around the world. It is obvious that the driving force is both economical and provides convenience for not only commercial applications but many products being developed for the military. Because it looks as though the world is ignoring the damaging health aspects of this technology that something needs to be done to allow its use but provide a way to remediate the damage. As a result, this invention has been created to provide the ability to coexist in a friendly manner with the EMF technology.

There are over 10,000 scientific papers and studies showing evidence of the mechanism of damage leading to health problems in living organisms. The first element of damage is how EMF saturation in our environment has caused us to lose the ability to have healthy negative ions at the surface of the earth. We know that healthy negative ions provide the grounding (also known as “earthing”) that all living things on earth require for optimal health. The saturation of electromagnetic frequencies that generate unhealthy negative ions extend about 3 feet downward into the earth from the surface. One major effect the electromagnetic frequencies are causing is the increase in free radicals in the body. A very good example of what this free radical damage can do to the body is Parkinson's disease. The nigral cells at the top of the brain stem are white when a person is born and over 18 years these white cells turn to a gray color. These cells produce dopamine. There are approximately 400,000 nigral cells at birth and when you lose 240,000 of these cells, you develop the symptoms of Parkinson's disease. Each year a healthy person will lose approximately 2,400 of these nigral cells due to free radical damage from normal metabolism. If you do a simple mathematical calculation, you will see that losing 2,400 of these cells each year, by the time you are 100 years old, you will have lost 240,000 cells. So, anything that generates more free radicals in the body will accelerate the rate at which the nigral cells are destroyed and the sooner a person will get Parkinson's disease.

The standard definition of a free radical is: any atom, molecule or ion that has one or more unpaired electrons in its orbit. A radical can have a net charge of negative, positive or no net charge (neutral). The term radical is synonymous with the term free radical. It is this very fact of the unpaired electron that drives the free radical to do its damage. A free radical is always looking for that “other electron” to become paired again. A free radical will inevitably take another atom, molecule or ion for that electron. The entity that was used is damaged and in fact becomes a free radical itself because it now needs to replace the electron that was taken. This is how free radicals do their damage.

Dirty electricity is another form of electromagnetic frequencies that needs to be acknowledged along with microwave discussions. Dirty electricity is erratic spikes and surges of electrical energy traveling along power lines and building wiring such as in homes, schools, and businesses today. Some common sources of dirty electricity include light dimmer switches, computers, laptops, televisions, video game systems, SMART meters and appliances, battery charging devices, blenders and mixers, multi-speed fans, fluorescent light bulbs and tubes, printers and scanners, music entertainment systems, cordless phone systems, Wi-Fi routers and modems, microwave ovens, hair dryers, solar power systems.

Dirty electricity has been with us since the invention of electricity and the development of commercially available appliances powered by electrical current. Many health problems can be associated with dirty electricity, but in today's world we have added microwave energy to the overall electromagnetic pollution, increasing EMF in the environment by over a quadrillion times in the last decade. The first case of myocardial infarction in America was recorded in 1923 and was considered rare. The direct current promoted by Edison did not create the dirty electricity problem. It was the alternating current from Tesla that did.

Our bodies contain 3 mechanisms that provide some protection from electrical damage. They are the P53 protein, glial cells, and the nuclear pore complex found in every cell. These elements are activated by electrical currents such as static electricity, lightning strikes, etc. The Wi-Fi microwave energy operates below this activation threshold level in our bodies, therefore making those natural protections useless against EMF. This issue relating to the amount of energy when discussing the safety of Wi-Fi brings to light the fact that Wi-Fi is far more dangerous than some other forms of EMF because it is this level of energy that flies below the threshold to activate tBA damage.

When we are subjected to these electrical assaults from EMF, every cell in the body of humans and animals contain voltage-gates that allow nutrients to move in and out of the cells and are affected in a way that damages our cells, especially that very structure that carries our chromosomes called the DNA. Dr. Martin Pall has identified this DNA damage in many peer reviewed scientific papers after studying this situation for over 18 years of his intensive research on the subject. When in an EMF environment, the voltage-gate calcium channel is opened, causing the cell to be flooded with calcium ions in excess of normal, thereby causing interruption in the manufacturer of nitric oxide. In addition, the breaking down of DNA structure causing DNA mutations. It is this mutation that is believed to be the basis for causing cancer in humans and animals.

The effect on vegetation of trees, flowers, weeds, and other vegetation when grown in an EMF environment then compared to the same plants grown in the remediated environment of this invention showing higher yields and better nutrient quality.

When dirty electricity and microwave frequencies are emitted from various sources, the waves strike the air and any structure in the environment causing the waves to become chaotic. It is this chaos that creates a major part of this problem. According to doctor Martin Pall.

Grounding, or earthing, is the electrically conductive contact of your body with the surface of the earth, such as when walking barefoot on grass. Grounding shows benefit for inflammation, the immune response, wound healing, and the prevention and treatment of chronic inflammatory and autoimmune diseases.

The earth carries an enormous negative charge. It&#39;s always electron-rich and can serve as a powerful and abundant supply of antioxidant and free-radical-busting electrons. Your body is finely tuned to “work” with the earth in the sense that there's a constant flow of energy between your body and the earth.

Grounding has been shown to relieve pain, reduce inflammation, improve sleep, enhance well-being, and much, much more. Unfortunately, many humans, animals, and vegetation living in developed countries are rarely grounded anymore.

More than a dozen researchers have conducted research on the physiological effects of grounding. Many studies have been published in peer-reviewed journals showing its benefit for fighting inflammation, improving the immune response, wound healing, and the prevention and treatment of chronic inflammatory and autoimmune diseases.

It is believed that mobile electrons create an antioxidant microenvironment around the injury repair field, slowing or preventing reactive oxygen species (ROS) delivered by the oxidative burst from causing ‘collateral damage’ to healthy tissue, and preventing or reducing the formation of the so-called “inflammatory barricade.”

It has been found that free radicals that leak into the healthy tissue will immediately be electrically neutralized when coming in contact with healthy negative ions. This occurs because the electrons are negative, while the free radicals are positive, so they cancel each other out.

According to Dr. Stephen Sinatra, a prominent cardiologist, inflammation thrives when your blood is thick and you have a lot of free radical stress, and a lot of positive charges in your body. Grounding effectively alleviates inflammation because it thins your blood and infuses you. Thus, a present need exists for a device capable of efficiently earthing a large area all at once.

### BRIEF SUMMARY OF THE INVENTION

It is an objective of the present invention to provide systems that allow for generating a healthy energy vortex within a space through an earthing effect, as specified in the independent claims. Embodiments of the invention are given in the dependent claims. Embodiments of the present invention can be freely combined with each other if they are not mutually exclusive.

The present invention features a system for generating a healthy energy vortex through an earthing effect. The system may comprise a plurality of copper-clad earthing poles disposed underground about a perimeter of an area. The area may be a building or a similar structure. Each earthing pole of the plurality of earthing poles may be completely submerged underground, may extend underground to contact a plurality of healthy negative ions underground, may not contact a separate structure such as a pipe or foundation, and each adjacent pair of earthing poles of the plurality of earthing poles may be separated by a distance suitable for generating the healthy energy vortex through the earthing effect. In some embodiments, each earthing pole of the plurality of earthing poles may further comprise a cap of grounding enhancement material disposed over a top of the earthing pole to more effectively generate the healthy energy vortex. In some embodiments, the system may further comprise a wire disposed under the cap of grounding enhancement material of each earthing pole of the plurality of earthing poles, such that the wire connects the plurality of earthing poles along the perimeter of the area to create a continuous bond between the perimeter of earthing poles to more effectively generate the healthy energy vortex. In some embodiments, the system may further comprise a plurality of magnet pairs disposed underground outside of the perimeter of the area encompassed by the plurality of earthing poles to more effectively generate the healthy energy vortex.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a side view of a plurality of magnet pairs and a plurality of underground earthing poles capped by a grounding enhancement material and connected by a wire.

FIG. 2 shows a top view of a plurality of magnet pairs and a plurality of earthing poles connected by a wire encompassing a perimeter of a building,

### DETAILED DESCRIPTION OF THE INVENTION

Following is a list of elements corresponding to a particular element referred to herein:

100 earthing pole

110 grounding enhancement material

120 wire

200 magnet

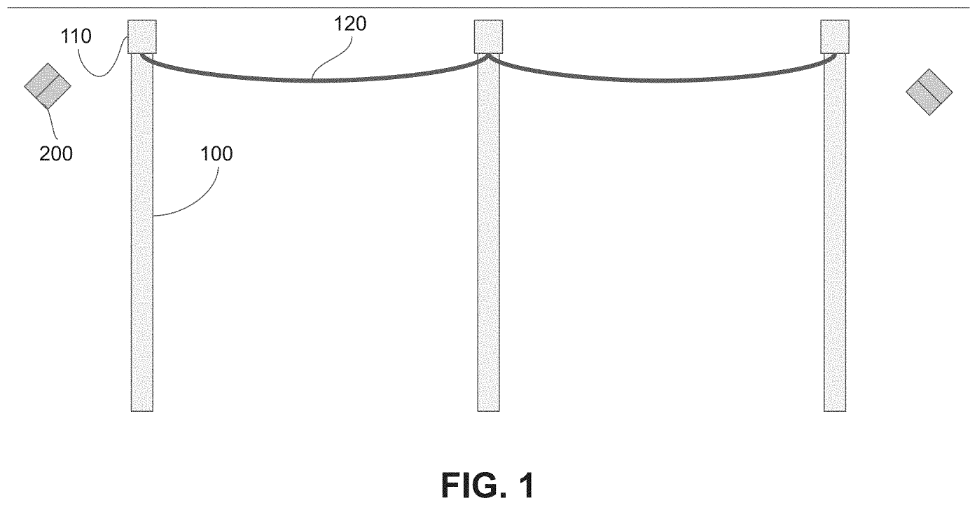

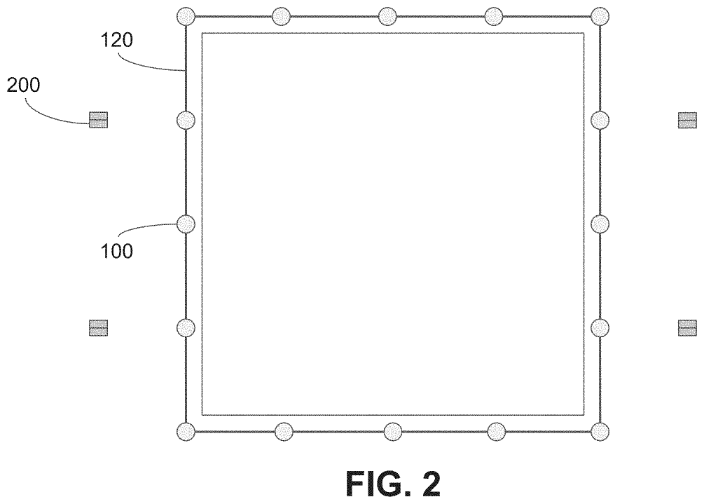

Referring now to FIGS. 1-2, the present invention features a system for generating a healthy energy vortex through an earthing effect. The system may comprise a plurality of copper-clad earthing poles disposed underground about a perimeter of an area. The area may be a building or a similar structure. Each earthing pole (100) of the plurality of earthing poles may be completely submerged underground with no part of the earthing pole (100) above-ground. Each earthing pole (100) of the plurality of earthing poles may contact a plurality of healthy negative ions underground. Each earthing pole (100) of the plurality of earthing poles may not contact a separate structure, such as a pipe or foundation. Each adjacent pair of earthing poles of the plurality of earthing poles may be separated by a distance suitable for generating the healthy energy vortex through the earthing effect. In some embodiments, each earthing pole (100) of the plurality of earthing poles may further comprise a cap of grounding enhancement material (110) disposed over a top of the earthing pole (100) to more effectively generate the healthy energy vortex. In some embodiments, the system may further comprise a wire (120) disposed under the cap of grounding enhancement material (110) of each earthing pole (100) of the plurality of earthing poles, such that the wire (120) connects the plurality of earthing poles along the perimeter of the area. The wire (120) may comprise a bare copper wire and the wire (120) may create a continuous bond between the perimeter of earthing poles to more effectively generate the healthy energy vortex.

In some embodiments, the system may further comprise a plurality of magnet pairs disposed underground outside of the perimeter of the area encompassed by the plurality of earthing poles. Each magnet pair (200) of the plurality of magnet pairs may be oriented at a 30 to 60 degree angle towards an inner corner of the perimeter of the area. Each magnet pair (200) of the plurality of magnet pairs may be oriented at a 45 degree angle towards an inner corner of the perimeter of the area. Each magnet pair (200) of the plurality of magnet pairs is disposed 1 to 12 inches away from the perimeter defined by the plurality of earthing poles. Each magnet pair (200) of the plurality of magnet pairs may be disposed 1 to 15 feet away from the perimeter defined by the plurality of earthing poles. A location of each magnet pair (200) and a quantity of the plurality of magnet pairs may be determined based on testing an electromagnetic field of the area for an ideal location and quantity of magnet pairs. Each magnet pair (200) of the plurality of magnet pairs may be disposed 9 to 12 inches underground. In some embodiments, each earthing pole (100) of the plurality of earthing poles may be 3 to 10 feet in length. Each earthing pole (100) of the plurality of earthing poles may be 8 feet in length. In some embodiments, each earthing pole (100) of the plurality of earthing poles may be ½ to 1 inch in diameter. Each earthing pole (100) of the plurality of earthing poles may be ⅝ inches in diameter. In some embodiments, each adjacent pair of earthing poles of the plurality of earthing poles may be separated by a distance of 12 to 20 feet. Each adjacent pair of earthing poles of the plurality of earthing poles may be separated by a distance of 16 feet or less.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A system for generating a healthy energy vortex through an earthing effect, the system comprising:
a. a plurality of copper-clad earthing poles disposed underground to define a perimeter of an area,
wherein each earthing pole (100) of the plurality of earthing poles is completely submerged underground,
wherein each earthing pole (100) of the plurality of earthing poles contacts a plurality of healthy negative ions underground,
wherein each earthing pole (100) of the plurality of earthing poles does not contact a separate structure,
wherein each adjacent pair of earthing poles of the plurality of earthing poles is separated by a distance suitable for generating the healthy energy vortex through the earthing effect.

2. The system of claim 1, wherein each earthing pole (100) of the plurality of earthing poles further comprises a cap of grounding enhancement material (110) disposed over a top of the earthing pole (100).

3. The system of claim 2 further comprising a wire (120) disposed under the cap of grounding enhancement material (110) of each earthing pole (100) of the plurality of earthing poles, such that the wire (120) connects the plurality of earthing poles along the perimeter of the area.

4. The system of claim 3, wherein the wire (120) comprises a bare copper wire.

5. The system of claim 1 further comprising a plurality of magnet pairs disposed underground outside of the perimeter of the area defined by the plurality of earthing poles.

6. The system of claim 5, wherein each magnet pair (200) is disposed 1 to 12 inches away from the perimeter defined by the plurality of earthing poles.

7. The system of claim 5, wherein each magnet pair (200) is disposed 1 to 15 feet away from the perimeter defined by the plurality of earthing poles.

8. The system of claim 5, wherein each magnet pair (200) of the plurality of magnet pairs is oriented at a 30 to 60 degree angle towards an inner corner of the area.

9. The system of claim 5, wherein each magnet pair (200) of the plurality of magnet pairs is oriented at a 45 degree angle towards an inner corner of the area.

10. The system of claim 5, wherein a size of the plurality of magnet pairs is determined based on testing an electromagnetic field of the area.

11. The system of claim 5, wherein each magnet pair (200) of the plurality of magnet pairs is disposed 9 to 12 inches underground.

12. The system of claim 1, wherein each earthing pole (100) of the plurality of earthing poles is 3 to 10 feet in length.

13. The system of claim 1, wherein each earthing pole (100) of the plurality of earthing poles is 8 feet in length.

14. The system of claim 1, wherein each earthing pole (100) of the plurality of earthing poles is ½ to 1 inch in diameter.

15. The system of claim 1, wherein each earthing pole (100) of the plurality of earthing poles is ⅝ inches in diameter.

16. The system of claim 1, wherein each adjacent pair of earthing poles of the plurality of earthing poles is separated by a distance of 12 to 20 feet.

17. The system of claim 1, wherein each adjacent pair of earthing poles of the plurality of earthing poles is separated by a distance of 16 feet or less.
